(19) United States
(12) Patent Application Publication
(10) Pub. No.: US 2023/0165785 A1
(43) Pub. Date: Jun. 1, 2023

(54) METHOD FOR OBTAINING AN EXTRACT OF SANDALWOOD, COMPOSITIONS COMPRISING SAME AND COSMETIC USES THEREOF

(71) Applicants: ISP INVESTMENTS LLC, Wilmington, DE (US); JAFER ENTERPRISES R&D, S.L.U, GRANOLLERS, BARCELONA (ES)

(72) Inventors: Catherine GONDRAN, BELLENAVES (FR); MUR Ludivine, PEGOMAS (FR); Isabelle IMBERT, CANNES (FR); Erell LE BORGNE, MOUGINS (FR); Sébastien GARNIER, LE ROURET (FR)

(73) Assignees: ISP INVESTMENTS LLC, Wilmington, DE (US); JAFER ENTERPRISES R&D, S.L.U, GRANOLLERS, BARCELONA (ES)

(21) Appl. No.: 17/922,749

(22) PCT Filed: May 4, 2021
(86) PCT No.: PCT/EP2021/061634

§ 371 Date: Nov. 1, 2022

(30) Foreign Application Priority Data
May 19, 2020 (FR) FR2005085

(51) Int. Cl. A61K 8/9789; A61Q 19/02; A61Q 7/00

(52) U.S. Cl. CPC A61K 8/9789; A61Q 19/02; A61Q 7/00

(57) ABSTRACT

The invention relates to the method for obtaining an extract of sandalwood (Santalum album) by extraction using a supercritical fluid and a co-solvent chosen from primary or secondary alcohols. The invention also relates to a crude extract of sandalwood comprising from 10 to 70% of volatile compounds and from 30 to 90% of semi- to non-volatile compounds. The invention also relates to cosmetic compositions comprising the solubilised form of such an extract, as well as to the cosmetic uses of such compositions for care of the skin, scalp and appendage

### TECHNICAL FIELD

The present invention relates to the field of cosmetics and more particularly to the field of active ingredients used in the formulation of skin care compositions. The invention relates to an extract of sandalwood (Santalum album) comprising from 10 to 70% of volatile compounds and from 30 to 90% of semi-volatile to non-volatile compounds, to the method for obtaining this extract, to the cosmetic compositions comprising same, and, lastly, to the cosmetic uses of such compositions for care of the skin, the scalp and the appendages.

White Sandalwood (Technical Background of the Invention Santalum album L.), also known as Indian sandalwood, is a shrub of the Sandalaceae family, native to the Indian peninsula and South-East Asia, and has long been cultivated in Australia.

Sandalwood is mainly used in perfumery and cosmetics, as an essential oil produced by distillation of the trunk and other parts of the tree (branches, roots) or by more complex extraction techniques.

The main compounds in sandalwood essential oils are sesquiterpene alcohols (content over 90%), in particular (Z)-alpha-santalol (up to 50%) and (Z)-beta-santalol (up to 30%). (Z)-alpha-santalol has a woody cedarwood-like odour, while (Z)-beta-santalol imparts the characteristic notes of sandalwood (Brocke, C.; Eh, M.; Finke, A., Recent Developments in the Chemistry of Sandalwood Odorants, Chem. Biodiv. 2008, 5, 1000-1010).

Sandalwood essential oil contains other sesquiterpene alcohols such as (E)-beta-curcumen-12-ol (2%), (Z)-gamma-bisabolen-12-ol (2%), (6R,7S)-iso-beta-bisabolol (1%), (6S,7S)-iso-beta-bisabolol, epi-alpha-bisabolol and (Z)-alpha-trans-bergamotol (6%) (Baldovini, N.; Delasalle, C.; Joulain, D., Phytochemistry of the heartwood from fragrant Santalum species: a review, Flavour Fragr. J. 2011, 26, 7-26).

Sandalwood is known and used in Ayurvedic medicine and aromatherapy in the treatment and prevention of a wide range of ailments, particularly to relieve anxiety, stress and depression (Kumar, R.; Anjum, N.; Tripathi, J. C., Phytochemistry and Pharmacology of Santalum album L.: A Review, World J. Pharm. Res. 2015, 4, 1842-1876; Setzer, W. N., Essential Oils and Anxiolytic Aromatherapy, Nat. Prod. Commun. 2009, 4, 1305-1316).

Sandalwood, especially through the presence of the sesquiterpene alpha-santalol, is also known for its anti-inflammatory, anti-oxidant, anti-viral and anti-bacterial activities, but also for its chemopreventive and anti-cancer properties (Santha, S.; Dwivedi, C. Anticancer effects of sandalwood (Santalum album), Anticancer Res. 2015, 35, 3137-3146).

Extraction methods for sandalwood using supercritical CO₂ are described in several documents. Document CN104232308 describes a method for extracting Santalum album roots. After freeze-drying and grinding by pulverisation, the plant material is extracted with supercritical CO₂ at a pressure between 30 and 35 MPa and a temperature between 50 and 65° C. Only the first two fractions are considered and combined to give the sandalwood extract.

It is also known from the work of Falconieri D. and colleagues that the recommended conditions for the extraction of plant material by supercritical CO₂ are: pressure of 90 bar, temperature of 50° C. (density CO₂=0.287 g/cm³) with a flow rate of CO₂ between 0.6 and 1.5 Kg/h (Extraction of Essential Oils from Natural Matrices, Acta Hortic. 2010 4 229-240).

Marongiu B. and colleagues describe a method for obtaining an essential oil by supercritical CO₂ from coarsely ground Santalum album wood using the following parameters: extraction temperature and pressure of 45° C. and 120 bar (density of CO₂=0.658 g/cm³), respectively, separator temperature and pressure of 15° C. and 20 bar, respectively, flow rate of CO₂ fixed at 1.5 Kg/h. These conditions make it possible to obtain a sandalwood essential oil with a yield of 1.9%, consisting mainly of alpha-santalol (46.1%), beta-santalol (20.4%), epi-beta-santalol (6.8%) and trans-alpha-bergamotol (5.4%) A comparison was made with the composition of an essential oil obtained by simple steam distillation (Extraction of Santalum album and Boswellia carterii Birdw. volatile oil by supercritical carbon dioxide: influence of some process parameters Flavour Fragr. J. 2006 21 718-724).

Nevertheless, the sandalwood extracts described in the prior art have a characteristic olfactory note and contain a non-negligible quantity of high-risk, volatile compounds, such as allergens. As the raw material used to produce the extract of the present invention is already depleted due to steam distillation, the extract developed is depleted of volatile compounds, and therefore in allergens, and has a much less intense olfactory note.

The extract of the invention is different from the sandalwood essential oils classically described in that it contains mainly semi- to non-volatile compounds detailed below:
phenolic compounds represented by a mixture of phenolic acids and aldehydes,
lignans
and more apolar compounds mainly represented by a mixture of acetylenic acids, fatty acids,
sesquiterpene derivatives of sandalwood, such as dimers and aliphatic esters of sandalwood, and
saponifiable compounds.

Sandalwood extracts are also known to have an effect on the skin, particularly in preventing the appearance of wrinkles and fine lines (KR101220903B1). The essential oil is also recommended for the care of dry or irritated skin, skin inflammation, or itching of the scalp.

The skin is an organ composed of several layers (dermis, epidermis and stratum corneum), which covers the entire surface of the body and ensures protective functions against external aggressions, sensory, immune, metabolic, thermoregulatory or even barrier functions, limiting dehydration. In particular, the skin has a regeneration function of its barrier function when it is subjected to external aggressions, as well as antioxidant and detoxifying defence mechanisms, which are solicited in response to these aggressions. Maintaining the skin's barrier function is essential for maintaining skin integrity.

The appearance of the skin can be modified by internal alterations (intrinsic ageing, diseases, and hormonal changes such as pregnancy) or external factors (environmental factors, such as pollution, sunlight, pathogens, temperature variations, etc.). All these alterations affect not only the skin, but also the keratinous appendages such as hair, eyelashes, eyebrows, nails and hair.

The olfactory receptor system (including OR2AT4) in the skin has a role in epidermal cell regeneration, including stimulating epidermal cell renewal and migration (Denda M. Newly discovered olfactory receptors in epidermal keratinocytes are associated with proliferation, migration, and re-epithelialisation of keratinocytes. J Invest Dermatol. 2014 November; 134(11):2677-2679).

The skin and its appendages (hair follicle, sebaceous gland) are endowed with olfactory receptors including the OR2AT4 receptor associated with hair growth (Cheret J. et al. Olfactory receptor OR2AT4 regulates human hair growth. Nat Commun 9, 3624,2018).

New molecules acting on olfactory receptors are believed to have an effect on the skin's ability to regenerate and to detect external pollutants.

The inventors have thus demonstrated that a particular new sandalwood extract, obtained from depleted sandalwood by an extraction method allowing both volatile compounds and semi- to non-volatile compounds of variable polarities (phenolic and lipid compounds) to be extracted, has beneficial effects on the skin and hair.

It has now been shown that, due to its particular composition, sandalwood extract has a higher biological activity than sandalwood essential oil.

### SUMMARY OF THE INVENTION

The invention relates firstly to a method for obtaining an extract of sandalwood (Santalum album) comprising the following steps
a) adding between 30 and 50% water to the depleted sandalwood;
b) optionally, mixing the moistened sandalwood with 1 to 20% of an inert compound to improve the diffusivity of the extraction solvent,
c) performing an extraction using a fluid in the supercritical state chosen from carbon dioxide (CO₂) and xenon, in the presence of a polar co-solvent chosen from primary or secondary alcohols, or any mixture thereof,
d) evaporating the extract obtained in step c) to remove the co-solvent and to obtain a crude extract in paste form.

The invention relates secondly to a crude extract of depleted depleted sandalwood obtainable by the method of the invention, characterised in that it comprises from 10 to 70%, preferably from 12 to 23%, more preferably from 14 to 21%, and even more preferably from 16 to 19% of volatile compounds on the one hand and from 30 to 90%, preferably from 77 to 88%, more preferably from 79 to 86%, and even more preferably from 81 to 84% of semi- to non-volatile compounds on the other hand.

The invention also relates to a solubilised extract of depleted depleted sandalwood obtainable by the method of the invention, comprising from 0.5 to 1.5% of crude extract, solubilised in a solvent selected from saturated or unsaturated, linear or branched, fatty alcohol type solvents comprising from 8 to 30 carbons or glyceride type solvents or any mixture thereof.

The invention relates thirdly to a cosmetic composition comprising, as active agent, a solubilised extract of depleted sandalwood, at a concentration of between 0.001 and 1%, obtained according to the method of the invention, and a physiologically acceptable medium.

The invention relates fourthly to the cosmetic use of a composition comprising the extract of depleted sandalwood of the invention for the care of the skin, the scalp, and the appendages.

### BRIEF DESCRIPTION OF THE DRAWINGS

The following figures illustrate the advantages arising from the invention and the non-limiting embodiments presented in the description:

FIG. 1 Analytical comparison of the DEDL chromatographic profiles of a sandalwood essential oil as described in Example 3 and of the crude sandalwood extract according to Example 1.

FIG. 2 Analytical comparison of the UV chromatographic profiles at 300 nm of a sandalwood essential oil as described in Example 3 and of the crude sandalwood extract according to Example 1.

FIG. 3 Quantification of OR2AT4 immunostaining in ex vivo skin biopsies.

### DETAILED DESCRIPTION OF THE INVENTION

### Definitions

All of the terms used in the present description have the most widely known meaning unless otherwise stated. For the purposes of the invention the following terms are defined as follows:

The term “ depleted sandalwood” is defined as sandalwood (Santalum album) trunk, branch and root chips recovered after extraction of the essential oil by steam distillation and then dried.

A “polar co-solvent” means a solvent of higher polarity than CO₂ in the supercritical state, such as primary or secondary alcohols, or any mixture thereof.

A “glyceride solvent” means fatty acid esters and glycerol esters.

When a range of values is described, the bounds of that range shall be understood as explicitly including the upper and lower bounds of that range, as well as all intermediate values in the range. For example, a range of values between 1% and 10% should be understood to include 1%, 2%, 3%, 4%, 5%, 6%, 7%, 8%, 9% and 10%, and all decimal values between 1% and 10%.

Numerical percentage values are percentages by weight, i.e. the weight of a compound in relation to the total weight of the intended mixture, unless otherwise specified.

The compositions described in the present application may “comprise”, “consist of” or “consist substantially of” the essential compounds or optional ingredients.

The expression “consist substantially of” means that the composition or component may include additional ingredients, but only if the additional ingredients do not alter the basic or novel characteristics of the composition or use described in this application.

The term “cosmetically compatible solvent” means a solvent that is suitable for contact with the outer layers of the skin, scalp or appendages, without toxicity, irritation, undue allergic response or similar, or intolerance reaction, and at the concentrations used is proportionate to a reasonable benefit/risk ratio.

The term “active agent” means a compound capable of acting on skin functions, with the aim of maintaining skin integrity, or restoring it in the event of imbalance caused by external factors (UV, pollution, detergents, etc.) or during ageing, with the aim of improving the appearance of the skin.

### Extraction Method

The sandalwood extracts described in the prior art have an intense olfactory note, characteristic of this wood, and contain a non-negligible quantity of high-risk, volatile compounds, such as allergens.

The inventors wanted to develop an extract that did not have these characteristics. To do this, they chose to use a raw material different from native sandalwood (trunk, branches or roots).

To produce the extract of the present invention, the inventors chose to use sandalwood (Santalum album) chips recovered after extraction of the essential oil by distillation, and then dried. Thus, as the sandalwood is already depleted due to extraction of the essential oil by steam distillation, the extract obtained is depleted of volatile compounds. It therefore has a much less intense olfactory note and the allergen content is greatly reduced. Moreover, the use of this perfumery co-product limits the impact on the environment and on biodiversity and allows a better management of plant resources.

The extraction method of the present invention has been developed extensively in order to optimise the extraction parameters. In this context, the influence of the extraction temperature, of the percentage of inert compound for improving the diffusivity of the extraction solvent, of the percentage of wetting of the material, and also of the flow rate of the co-solvent has been studied.

The invention thus relates firstly to a method for obtaining an extract of sandalwood (Santalum album) comprising the following steps
a) adding between 30 and 50% water to the depleted sandalwood;
b) optionally mixing the depleted sandalwood obtained in step a) with 1 to 20% of an inert compound for improving the diffusivity of the extraction solvent;
c) performing an extraction using a fluid in the supercritical state chosen from carbon dioxide (CO₂) and xenon, in the presence of a polar co-solvent chosen from primary or secondary alcohols, or any mixture thereof;
d) evaporating the extract obtained in step c) to remove the co-solvent and thus to recover a crude extract in paste form.

In order to carry out step a), water is added to the depleted sandalwood to promote the permeation of the solvent into the interstitial phase of the wood to extract the extractable compounds. This step consists in adding extemporaneously between 30 and 50% of water to the depleted sandalwood.

The chips are very dry and porous and easily absorb the water that is added at this stage, leaving a moistened raw material.

Advantageously, dried, debarked sandalwood (Santalum album) trunk chips are used.

Advantageously, the depleted sandalwood can come from sandalwood (Santalum album) grown in Australia.

Step b) is not mandatory, but can improve the extraction method. In this step, the moistened depleted sandalwood obtained in step a) is mixed with 1-20% of an inert compound to improve the diffusivity of the extraction solvent and thus avoid the creation of preferential paths within the plant material. The inert compound is chosen from organic or inorganic compounds and preferably the inert compound is powdered cellulose.

Preferably, the inert compound is used at a concentration of 5 to 15%.

In the course of this description, the presence of the inert compound is not taken into account in the calculations of mass ratios and yields. The values given only take into account the plant raw material.

To carry out step c), a fluid in the supercritical state chosen from among carbon dioxide (CO₂) and xenon can be used. However, carbon dioxide is preferred.

For this step, a polar co-solvent is also used, to increase the polarity of the CO₂ in the supercritical state and thus to favour the extraction of polar compounds.

The co-solvent is chosen from solvents of higher polarity than CO₂ in the supercritical state, chosen from primary or secondary alcohols, or any mixture thereof.

Preferably the co-solvent is ethanol at a concentration of between 80 and 100% (v/v) in water, preferably between 90 and 100% (v/v) in water, and even more preferably at a concentration of 96% (v/v) in water.

The mass ratio of supercritical fluid, when the fluid is carbon dioxide, to the amount of raw material used (depleted sandalwood) is between 10 and 50, advantageously between 20 and 40, and preferably between 25 and 35.

The mass ratio of supercritical fluid (carbon dioxide) to co-solvent is between 0.050 and 0.080, advantageously between 0.055 and 0.075, and preferably between 0.060 and 0.070.

In step c), the extraction temperature is between 35 and 85° C., advantageously between 45 and 75° C., and preferably between 55 and 65° C.

The pressure within the extractor is between 90 and 1000 bar, preferably between 150 and 700 bar, and even more preferably between 250 and 400 bar.

To carry out step c), advantageously the mixture obtained in step a) or step b) is placed in a stainless steel cartridge and this cartridge is introduced into a supercritical fluid extractor. The solvent used for extraction is carbon dioxide.

In step d), a first crude extract of depleted sandalwood obtained in step c) is recovered and is evaporated under vacuum to completely remove the co-solvent. The evaporation temperature is at most 65° C. and the pressure below 90 mbar to allow good evaporation of the co-solvent.

At the end of step d) the extraction yield is preferably between 0.5 and 2%.

At this stage, the crude extract of depleted sandalwood in the sense of the invention is obtained, which is in paste form and comprises:
from 10 to 70% of volatile compounds;
from 30 to 90% of semi- to non-volatile compounds; including 0.35 to 3.5% syringaldehyde.

The extraction may then be continued with a step e) in which the crude extract obtained in step d) is solubilised in a saturated or unsaturated fatty alcohol solvent, linear or branched, comprising 8 to 30 carbons or a glyceride solvent, or any mixture thereof, preferably to obtain a crude extract concentration of between 0.5 and 1.5% by weight of the total weight of the solubilised extract.

Preferably, the fatty alcohol or glyceride solvent is selected from octyldodecanol, 2-hexyl decanol, oleic alcohol and miglyol (triglyceride mixture), or any mixture thereof.

Even more preferably, the solvent is octyldodecanol.

Advantageously, the extraction may be continued by an optional step f) in which the extract obtained in step d) or e) is purified. This step may be carried out by any technique known to a person skilled in the art and in particular by chromatography or by molecular distillation. This step may, for example, make it possible to standardise the extract.

### Extract

The extract of the invention differs from the sandalwood extracts obtained by supercritical CO₂ in the prior art due to the fact that the raw material used is constituted by the co-products of distillation of wood chips of Santalum album, thus a material depleted of volatile compounds. The method described above makes it possible to obtain an extract enriched in polar and apolar compounds. This extract has a wide chemical diversity in that it contains both a residual proportion of volatile compounds not extracted during steam distillation and semi- to non-volatile compounds of varying polarity.

The invention relates secondly to a crude extract of depleted sandalwood obtainable by the method according to the invention.

The invention also relates to a crude extract of depleted sandalwood obtained directly by the method according to the invention.

A “raw extract” of depleted sandalwood means the extract in paste form obtained in step d) of the method.

The crude extract of depleted sandalwood itself contains:
between 10 and 70% of volatile compounds (mainly sesquiterpene alcohols, mainly (Z)-alpha-santalol, (Z)-beta-santalol and derivatives of these compounds), preferably between 12 and 23%, more preferably between 14 and 21%, and even more preferably between 16 and 19%;
between 30 and 90% of semi- to non-volatile compounds, preferably between 77 and 88%, more preferably between 79 and 86%, and even more preferably between 81 and 84%. This fraction is mainly composed of lipidic compounds represented by a mixture of acetylenic acids, fatty acids as well as sesquiterpene derivatives of sandalwood including dimers and aliphatic esters. The presence, in smaller quantities, of phenolic acids and aldehydes and lignans is also observed.

In a very advantageous embodiment, the crude extract comprises 16% volatile compounds and 84% semi- to non-volatile compounds including 0.6% syringaldehyde.

The term “volatile compounds” means organic molecules that can easily pass into the gas phase at atmospheric pressure and room temperature. These are compounds that have a very low boiling point. They evaporate or sublimate easily from their solid or liquid form. Gas chromatography is therefore the technique of choice to analyse and detect them in the extract. They are mainly sesquiterpene alcohols, mainly (Z)-alpha-santalol and (Z)-beta-santalol.

The term “semi- to non-volatile compounds” means molecules which do not pass easily into the gaseous phase at atmospheric pressure and at room temperature due to a higher boiling point than in the case of volatile molecules. In the case of the extract under consideration, these are molecules of medium polarity belonging to the family of phenolic compounds (phenolic acids and aldehydes, lignans) and apolar molecules belonging to chemical families such as fatty acids, acetylenic acids, sesquiterpenes derived from sandalwood as well as saponifiable compounds detected in the extract.

Volatile compounds are analysed by gas chromatography (GC) coupled to a mass spectrometer (MS) and/or a flame ionisation detector (FID). Confirmation of identifications is made possible by comparing linear retention indices and mass spectra contained in libraries. Quantification by GC/FID is done by internal calibration using predicted and calculated response factors.

Semi- to non-volatile compounds are monitored by high-performance liquid chromatography (HPLC) coupled to a diode array UV detector (DAD) and a light scattering evaporative detector (LSED). Structural identifications are confirmed by nuclear magnetic resonance (NMR) examinations as well as high-resolution mass spectrometry (HRMS) analyses. Identification is also confirmed by injection of the standard if commercially available. The overall content of semi- to non-volatile compounds is estimated by subtracting the content of volatile compounds. Syringaldehyde, forming the majority of the medium polar compounds, is considered as a marker of the crude extract and was quantified with a diode array detector by external calibration.

The main molecules identified are:
In the volatile fraction, compounds of the sesquiterpene alcohol type, mainly (Z)-alpha-santalol and (Z)-beta-santalol.
In the semi- to non-volatile fraction, fatty acids, acetylenic acids, saponifiable compounds, sesquiterpene derivatives of sandalwood as well as aldehydes and phenolic acids and lignans.

The non-exhaustive list of compounds present in these two fractions is given in the following Table 1:

TABLE 1

| Fraction | Chemical family | Identification |
|---|---|---|
| Volatile fraction | Sesquiterpene alcohols, | (Z)-alpha-santalol |
| | sesquiterpenes | (Z)-beta-santalol |
| Semi- to non-volatile | Phenolic aldehydes, | Syringic acid |
| fraction | phenolic acids, lignans, | Syringaldehyde |
| | fatty acids, acetylenic acids, | Syringaresinol |
| | sesquiterpene derivatives of | Oleic acid |
| | sandalwood, saponifiable | Palmitic acid |
| | compounds | Alpha-santaldiol |

Some markers of the raw extract could be quantified. This contains in particular:
between 0.35 and 3.5% syringaldehyde, advantageously between 0.4 and 3%, and preferably between 0.45 and 2.5%;
between 1.3 and 2.3% (Z)-alpha-santalol, advantageously between 1.4 and 2.2%, and preferably 1.6 and 2.0%;
between 0.7 and 1.3% (Z)-beta-santalol, advantageously between 0.8 and 1.2% and preferably between 0.9 and 1.1%.

The invention also relates to a solubilised extract of depleted sandalwood obtainable by the method according to the invention.

The invention also relates to a solubilised extract of depleted sandalwood obtained directly by the method according to the invention.

The term “extract of depleted sandalwood” or “solubilised extract of depleted sandalwood” in the sense of the invention means the liquid extract obtained after solubilisation in step e) of the method.

The solvents used for the solubilisation step are selected from saturated or unsaturated, linear or branched, fatty alcohol solvents comprising from 8 to 30 carbons or glyceride solvents, or any mixture thereof.

Preferably the fatty alcohol or glyceride solvent is selected from octyldodecanol, 2-hexyl decanol, oleic alcohol and miglyol (triglyceride mixture), or any mixture thereof.

Even more preferably, the solvent is octyldodecanol.

The solubilised extract of depleted sandalwood advantageously consists of 0.5 to 1.5% by weight crude extract in relation to the total weight of the extract solubilised in octyldodecanol.

Preferably, the extract of depleted sandalwood consists of 1.0% by weight crude extract in relation to the total weight of the extract solubilised in octyldodecanol.

The solubilised extract of depleted sandalwood is in liquid form and contains a mixture of molecules with a wide range of polarities. Chromatographic analysis of the solubilised extract shows a syringaldehyde content of between 0.0035% and 0.035%.

### Cosmetic Compositions

The invention relates thirdly to a cosmetic composition comprising, as an active agent, an extract of depleted sandalwood according to the invention, and a physiologically acceptable medium.

A “physiologically acceptable medium” means a vehicle that is suitable for contact with the outer layers of the skin, scalp or appendages, without toxicity, irritation, undue allergic or similar response or intolerance reaction, and proportionate to a reasonable benefit/risk ratio.

Examples of physiologically acceptable media commonly used in the intended field of application are formulation aids such as solvents, thickeners, thinners, antioxidants, colouring agents, sunscreens, self-tanning agents, pigments, fillers, preservatives, perfumes, odour absorbers, essential oils, vitamins, essential fatty acids, surfactants, film-forming polymers etc.

Preferably, the composition according to the invention comprises the extract of depleted sandalwood obtainable by the method according to the invention in a concentration of from 0.001 to 1% by weight in relation to the total weight of the composition, preferably from 0.1% to 1%, and a physiologically acceptable medium.

The composition usable in accordance with the invention can be applied by any suitable route, in particular externally topically, and the formulation of the compositions will be adapted by a person skilled in the art.

Preferably, the compositions according to the invention are in a form suitable for topical application.

The expression “topical application” means applying or spreading a composition comprising the extract of depleted sandalwood of the invention on the surface of the skin, scalp, mucosa or appendages.

The term “skin” refers to the skin of the face, including the eye area and mouth, the nose, the forehead, the neck, the hands, but also the skin of the whole body, including the scalp.

The term “scalp” means the skin covering the skull, including the hair follicles and the inter-follicular skin spaces.

The term “appendages” refers to the keratinised skin appendages present in humans and animals, rich in keratin, and more particularly to hair, eyelashes, eyebrows and nails.

The compositions according to the invention are particularly suitable for topical application to healthy skin.

For the purposes of the present invention, healthy skin is defined as skin that is free of skin pathology.

The topical compositions for carrying out the invention may in particular be in the form of an aqueous, aqueous-alcoholic or oily solution, an oil-in-water emulsion, a water-in-oil emulsion, a multiple emulsion, a micro-emulsion, a nano-emulsion or any colloidal system that can be used in cosmetics; they may also be in the form of suspensions or powders suitable for application to the skin, mucous membranes, lips and/or hair.

These compositions may be more or less fluid and may also be in the form of a cream, lotion, milk, serum, ointment, gel, paste or foam. They may also be in solid form, such as a stick, or formulated to be compatible with aerosol delivery.

In all cases, a person skilled in the art will ensure that the adjuvants and their proportions are chosen in such a way as not to impair the advantageous properties sought of the composition according to the invention. These adjuvants may, for example, correspond to 0.01 to 20% of the total weight of the composition. When the composition according to the invention is an emulsion, the fatty phase may represent from 5 to 80% by weight and preferably from 5 to 50% by weight relative to the total weight of the composition. The emulsifiers and co-emulsifiers used in the composition are chosen from those conventionally used in the field under consideration. For example, they may be used in a proportion ranging from 0.3 to 30% by weight relative to the total weight of the composition.

In a particular embodiment the compositions can contain additional active agent(s) to enhance the effect of the sandalwood extract used in the invention.

The INCI Dictionary & Handbook (“International Nomenclature of Cosmetic Ingredients 13th Ed. 2010” published by the Personal Care Products Council, Inc., Washington, D.C.) describes a wide variety, without limitation, of cosmetic ingredients commonly used in the skin care industry, which are suitable for use as additional active agents in the compositions according to the present invention.

Non-limiting examples of these classes of additional active agents include:

anti-ageing agents, anti-wrinkle agents, moisturising agents, softening agents, keratolytic or desquamating agents, anti-seborrhoeic agents, anti-dandruff agents, agents modulating skin cell differentiation or proliferation, agents modulating skin pigmentation, self-tanning agents, anti atmospheric pollution agents, anti-glycation agents firming agents, aquaporin synthesis stimulating agents, lipid and stratum corneum components (ceramides, fatty acids) synthesis stimulating agents, adipocyte proliferation stimulating agents, glycosaminoglycan synthesis stimulating agents, DNA repairing agents, DNA protecting agents, agents for the treatment and/or care of sensitive skin, firming agents, anti-stretch mark agents, astringent agents, dermo-alpha-santalol agents, cytokine growth factors, agents acting on capillary circulation and/or microcirculation, agents inhibiting vascular permeability, agents acting on cellular metabolism, agents for improving the dermal-epidermal junction, agents inducing head hair and/or other hair growth, lipolysis stimulating agents, slimming agents, anti-cellulite agents, sunscreens, agents capable of reducing or treating bags under the eyes, and mixtures thereof, as long as they are physically and chemically compatible with the other ingredients of the composition and especially with the active ingredients of the present invention.

Furthermore, the nature of these additional active agents must not unacceptably alter the benefits of the active ingredients of the invention. These additional active agents can be synthetic or natural, such as plant extracts, or can be from a biofermentation process.

Such additional active agents can also be selected, according to their chemical composition, from the group comprising: amino sugars, glucosamine, D-glucosamine, N-acetyl-glucosamine, N-acetyl-D-glucosamine, mannosamine, N-acetyl mannosamine, galactosamine, N-acetyl galactosamine, vitamin B3 and its derivatives, niacinamide, sodium dehydro-acetate, dehydroacetic acid and its salts, phytosterols, salicylic acid compounds, hexamidines, dialkanoyl dihydroxyproline compounds, extracts and derivatives of soya, equol, isoflavones, flavonoids, phytantriol, farnesol, geraniol, bisabolol, peptides and their derivatives, di-, tri-, tetra-, penta-, and hexapeptides and their derivatives, lys-thr-thr-lys-ser, palmitoyl-lys-thr-lys-ser, carnosine, N-acyl amino acid compounds, retinoids, retinyl propionate, retinol, retinyl palmitate, retinyl acetate, retinal, retinoic acid, water-soluble vitamins, ascorbates, vitamin C, ascorbyl glucoside, ascorbyl palmitate, magnesium ascorbyl phosphate, sodium ascorbyl phosphate, vitamins and their salts and derivatives, provitamins and their salts and derivatives, ethyl panthenol, vitamin A and its derivatives, vitamin B and its derivatives, vitamin B1, vitamin B2, vitamin B6, vitamin B12, vitamin E, vitamin F, vitamin K and its derivatives, pantothenic acid pantothenyl ethyl ether, panthenol and its derivatives, ethyl panthenol, dexpanthenol, biotin, amino acids and their salts and derivatives, water-soluble amino acids, asparagine, alanine, indole, glutamic acid, water-insoluble vitamins, beta-ionol, cedrol, and their derivatives, water-insoluble amino acids, tyrosine, tryptamine, particulate materials, butylated hydroxytoluene, butylated hydroxyanisole, allantoin, tocopherol nicotinate, tocopherol, tocopherol esters, palmitoyl-gly-his-lys, phytosterol, hydroxy acids, glycolic acid, lactic acid, lactobionic acid, keto acids, pyruvic acid, phytic acid, lysophosphatidic acid, stilbenes, cinnamates, resveratrol, kinetin zeatin, dimethylaminoethanol, natural peptides, soy peptides, acid sugar salts, manganese gluconate, zinc gluconate, piroctone olamine, 3,4,4′-trichlorocarbanilide, triclocarban, zinc pyrithione, hydroquinone, kojic acid ascorbic acid, magnesium ascorbyl phosphate, ascorbyl glucoside, pyridoxine, aloe vera, terpene alcohols, allantoin, bisabolol, dipotassium glycyrrhizinate, glycerol acid, sorbitol, pentaerythritol, pyrrolidone and its salts, dihydroxyacetone, erythrulose, glyceraldehyde, tartaraldehyde, clove oil, menthol, camphor, eucalyptus oil, eugenol, menthyl lactate, witch hazel distillate, eicosene copolymer and vinyl pyrrolidone, iodopropyl butylcarbamate, polysaccharide, essential fatty acid, salicylate, glycyrrhetinic acid, carotenoids, ceramides and pseudoceramides, complex lipid, oils in general of natural origin such as shea butter, apricot oil, evening primrose oil, prune oil, palm oil, monoi oil, kahai oil, hydroquinone, HEPES, procysteine, O-octanoyl-6-D-maltose, disodium salt of methyl glycine diacetic acid, steroids such as diosgenin and DHEA derivatives, DHEA dehydroepiandrosterone and/or a chemical or biological precursor or derivative, N-ethylcarbonyl-4-para-aminophenol, alpha hydroxy acids, beta hydroxy acids, moisturisers, epidermal hydrolytic enzymes, plant extracts, phytohormones, yeast extracts, a metalloproteinase inhibitor, enzymes, enzyme inhibitors, enzyme inducers, coenzymes, chelating agents, plant extracts and plant derivatives, essential oils, marine extracts, agents from a biofermentation and/or biotechnology process, mineral salts, cell extracts

The following can be mentioned by way of further examples:
peptides commercially known as MATRIXYL®, ARGIRELINE®, CHRONOGEN™, LAMINIXYL IS™, PEPTIDE Q10™, COLLAXYL™ (patent FR2827170, ASHLAND®), PEPTIDE VINCI 01™ (patent FR2837098, ASHLAND®), PEPTIDE VINCI 02™ (patent FR2841781, ASHLAND®), ATPeptide™ (patent FR2846883, ASHLAND®) or the synthetic peptide with the sequence Arg-Gly-Ser-NH2, marketed under the name ATPeptide™ by ASHLAND®;
Artemia salina extract, marketed under the name GP4G™ (FR2817748, ASHLAND®);
plant peptide extracts such as flax extracts (Lipigenin™, patent FR2956818, ASHLAND®), extracts of soya, spelt, vine, rape, flax, rice, maize, pea, cocoa;
yeast extracts, for example Dynagen™, (patent FR2951946, ASHLAND®) or Actopontine™ (patent FR2944526, ASHLAND®);

### Uses

The invention further relates to the cosmetic use of a composition comprising the extract of depleted sandalwood of the invention for care of the skin, scalp and appendages.

The cosmetic uses according to the present invention relate to cosmetic treatment methods by topical application to healthy skin.

The invention further relates to the cosmetic use of a composition according to the invention to improve the appearance of the skin, to combat the signs of skin ageing, to improve the ability of the skin to detect external pollutants, and to restore the skin's lipid barrier function.

The invention also relates to the cosmetic use of a composition according to the invention to increase the expression of olfactory receptors (OR2AT4) in the skin.

The invention further relates to the cosmetic use of a composition according to the invention to lighten the skin.

The invention further relates to the cosmetic use of a composition according to the invention to promote hair growth.

The expression “detect external pollutants” refers to the ability of the skin to trigger its defence mechanisms to reduce the effects of exposure to any type of atmospheric pollutant, including in particular odorous molecules of the volatile organic compound type.

The expression “improve the appearance of the skin” means reducing the damage caused by environmental stresses in order to restore skin function and limit visible damage to the skin, such as signs of ageing or skin sensitivity.

The term “skin sensitivity” means the reaction of healthy skin to various aggressions, with signs of discomfort such as tingling, heating or tightness of the skin of the face or scalp and possible visible reactions such as redness.

The term “signs of skin ageing” means changes in the external appearance of the skin due to ageing, chosen from wrinkles and fine lines, creases, bags under the eyes, dark circles, withering, loss of elasticity, firmness and/or tone of the skin, irregularity of the skin texture or complexion, but also all internal modifications of the skin which do not systematically result in a modified external appearance such as thinning of the skin, or all internal degradations of the skin following environmental stresses (or external aggressions).

The expression “improve the lipid barrier function or skin barrier function” means that the protective properties of the skin against external aggression are improved in order to maintain skin integrity.

The extract of depleted sandalwood of the invention was tested on the expression of olfactory receptors (OR2AT4) in the skin.

Odour perception involves olfactory (or odorant) receptors on the nasal epithelium. The activation of these receptors by odorant molecules leads to specific sensations, which can serve as warning or communication signals. Olfactory receptors belong to the family of G-protein coupled receptors. Their activation by an odorant molecule induces a calcium signal, indicating their functionality. Apart from the nasal epithelium, ectopic expression of certain types of olfactory receptors has been reported in different organs including the skin (Denda M. Newly discovered olfactory receptors in epidermal keratinocytes are associated with proliferation, migration, and re-epithelialisation of keratinocytes. J Invest Dermatol. 2014 November; 134(11):2677-2679). Indeed, during evolution, some species have retained olfactory receptors expressed on their skin surface. In general, they are involved in the recognition of environmental signals. Among these, OR2AT4-type receptors have been studied in greater detail with regard to their role in human skin. These receptors have the synthetic molecule Sandalore as their ligand and play a role in the skin regeneration process, by intervening in the proliferation and migration of keratinocytes (Busse D. et al. A synthetic sandalwood odorant induces wound-healing processes in human keratinocytes via the olfactory receptor OR2AT4. J Invest Dermatol. 2014 November; 134(11):2823-2832). Expression of OR2AT4-like receptors has also been reported in hair follicles, where they are associated with the hair growth phase (Chéret J. et al. Olfactory receptor OR2AT4 regulates human hair growth. Nat Commun 9, 3624,2018). Other receptor types have been described in the skin, including keratinocytes and melanocytes (Wojcik S, Weidinger D, Stander S, Luger T, Hatt H, Jovancevic N. Functional characterisation of the extranasal OR2A4/7 expressed in human melanocytes. Exp Dermatol. 2018 ;27(11):1216-1223).

### EXAMPLES

The present invention is illustrated by the following non-limiting examples:

### Example 1: Preparation of an Extract of Depleted Sandalwood

For the examples, the white sandalwood (Santalum album) trees were grown in Australia.

As white sandalwood is not native to Australia, and the raw material was sourced from private plantations not proprietary of Commonwealth land, no Part 8A permit under the EPBC Act (Environment Protection and Biodiversity Conservation Act 1999, governing, inter alia, access to genetic resources and benefit sharing—ABS) was required.

The plant part used was constituted by the depleted , bark-free chips of the trunk of the Santalum album tree, i.e. recovered after steam distillation. During the development of the method, several parameters were studied, namely the wetting rate of the plant material, the percentage of added cellulose, the extraction temperature and the co-solvent flow rate.

Table 2 shows some simulated results obtained from the experimental design conducted to optimise the extraction parameters:

TABLE 2

| | | Co- | | | Simulated content of |
|---|---|---|---|---|---|
| | Extraction | solvent | | Simulated | phenolic compound (% in |
| % | temperature | flow rate | Water | extraction | gallic acid eq. in relation |
| cellulose | (° C.) | (ml/min) | (%) | yield (%) | to the primary material) |
| 0 | 40 | 10 | 60 | 0.68 | 0.00170 |
| 0 | 60 | 15 | 50 | 0.98 | 0.00166 |
| 10 | 55 | 15 | 50 | 1.06 | 0.00192 |
| 10 | 60 | 20 | 40 | 1.32 | 0.00176 |

The extraction conditions were selected in order to obtain a satisfactory extraction yield while ensuring a beneficial chemical diversity of the extract, in particular with a correct content of phenolic compounds. The preferred extraction parameters were as follows:

The chips were moistened with 40% water (% by weight), mixed with 10% powdered cellulose, and then placed in a stainless steel cartridge. This cartridge was introduced into a supercritical fluid extractor, such as the Separex SFE 5 extractor. The extraction solvent used was carbon dioxide in the supercritical state at a flow rate of 15 Kg/h complemented by 96% ethanol in water (volume/volume) as a polar co-solvent at a flow rate of 20 ml/min. The mass ratio of carbon dioxide to wetted plant was 30 and that of carbon dioxide to co-solvent was 0.065. The pressure and temperature in the extractor were 300 bar and 60° C., respectively. The pressure and temperature in the separator were 55 bar and 35° C., respectively. The extract thus obtained was evaporated under vacuum up to total evaporation of the ethanol (pressure below 90 mbar at a water bath temperature of 65° C.). The extraction yield was 1.2%.

The crude extract obtained was in the form of a paste.

The crude extract was solubilised in agro-sourced octyldodecanol to obtain a solubilised extract appearing as a clear and fluid solution containing 1.0% crude extract of Santalum album chips.

### Example 2: Characterisation of the Crude Extract of Depleted Sandalwood Obtained According to Example 1

The non-exhaustive list of compounds present in each of the fractions is detailed in Table 3 below:

TABLE 3

| Chemical family | Identification |
|---|---|
| Volatile fraction (sesquiterpene | alpha-santalene |
| alcohols, sesquiterpenes) | beta-santalene |
| | alpha-bergamotene |
| | beta-epi-santalene |
| | curcumen AR |
| | (Z)-alpha-santalol |
| | (Z)-beta-santalol |
| Semi- to non-volatile fraction (phenolic | Syringaldehyde |
| aldehydes, phenolic acid, lignan, fatty | Sinapaldehyde |
| acid, acetylenic acid, sesquiterpene | Vanillic acid |
| derivative of santalol, saponifiable) | Syringic acid |
| | Sinapic acid |
| | Oleic acid |
| | Palmitic acid |
| | linoleic acid |
| | linolenic acid |
| | alpha-santaldiol |
| | 2,12,13-trihydroxy- |
| | 10-campherene |
| | aliphatic ester of |
| | alpha-santalol |
| | dimer of santalol |

Some markers of the raw extract could be quantified. The extract contained in particular:
0.6% syringaldehyde;
1.8% (Z)-alpha-santalol;
1.0% (Z)-beta-santalol;
16% volatile fraction;
84% semi- to non-volatile fraction.

### Example 3: Production of an Essential Oil Extract of Sandalwood

In order to perform a comparative analysis, an essential oil extraction of sandalwood (not depleted) was performed in a conventional way, by steam distillation. The trunk without the bark, branches and roots of Santalum album were dried and then coarsely ground into chips, which were placed in separate stills and then passed through a stream of steam; this steam released the volatile molecules or essential oil, which were carried away by the steam and condensed in the condenser. Since the essential oil is less dense than water and is not or only slightly water-soluble, it was collected at the outlet in a decanter called the essencier. The water still containing traces of the essential oil is called hydrolate.
The essential oil is obtained by making a communal mixture from the three oils previously made. It is a pale yellow to yellow liquid.

### Example 4: Identification of Major Phytochemical Differences Between an Essential Oil and the Extract Obtained in Example 1.

The crude extract of depleted sandalwood obtained in Example 1 was compared to an essential oil of Santalum album as prepared in Example 3 by high-performance liquid chromatography (HPLC) analysis coupled to a diode array detector and a DEDL detector of solutions of the same concentration of these samples. The chromatographic analysis was performed on a Core-Shell C18 column in an elution gradient using acidified mobile phases consisting of a mixture of water/ACN/IPA for lane A and ACN/IPA/MeOH for channel B.

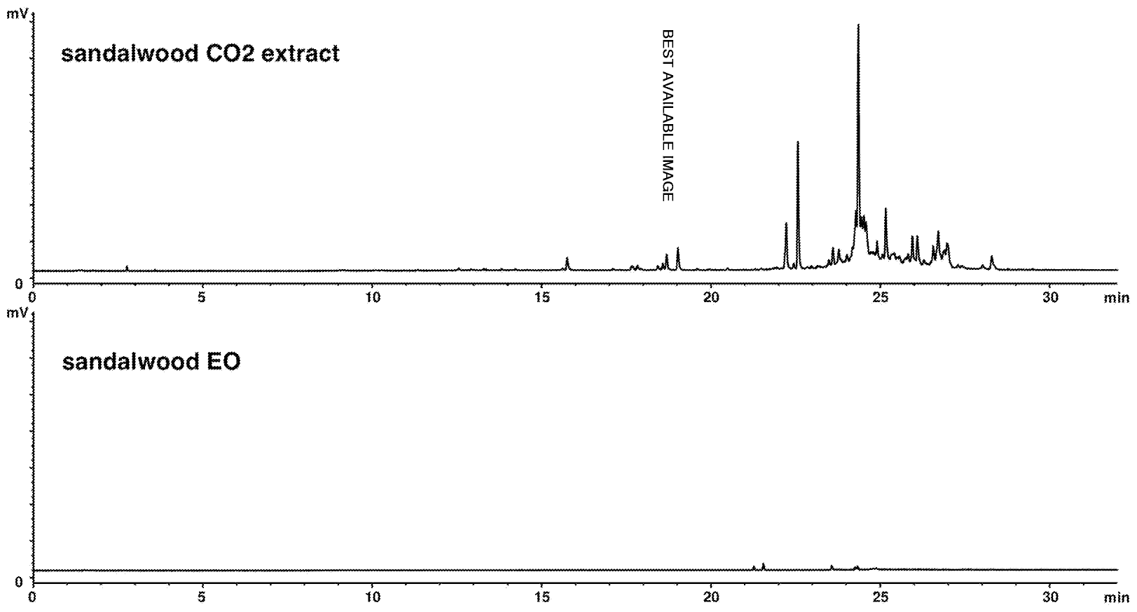

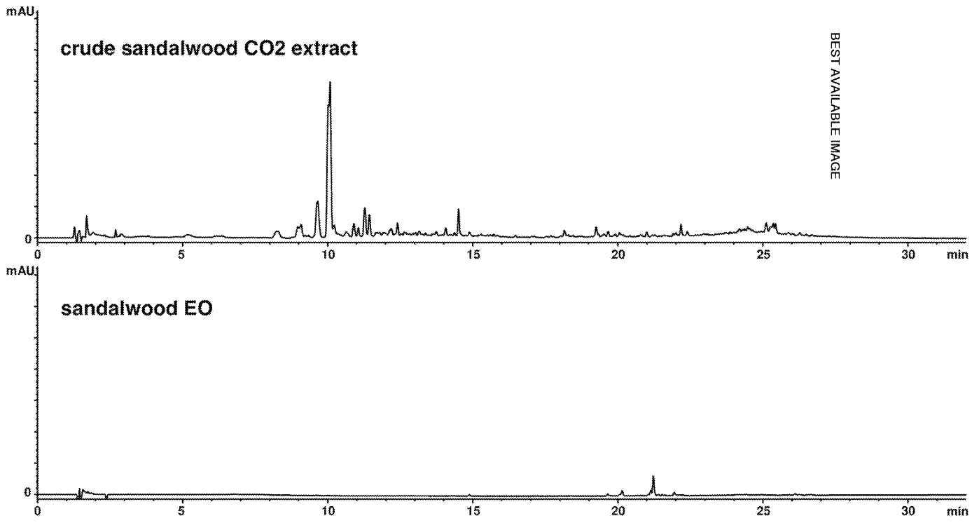

FIGS. 1 and 2 show the DEDL and UV chromatographic profiles at 300 nm of sandalwood essential oil as described in Example 3 and of the crude extract of depleted sandalwood according to Example 1, analysed in HPLC/UV-DEDL on a Core-Shell C18 column with gradient elution (0-5 min 100% A, 5-22 min change from 100% A to 100% B then 22-32 min 100% B-A: H₂O/ACN/IPA/HCO₂H 95/2.5/2.5/0.1 (v/v/v/v) and B: IPA/ACN/MeOH/HCO2H 40/40/20/0.1 (v/v/v/v). The y-axis shows the detector response in mV for the DEDL profile and in mAU for the diode array profile. In both cases, the abscissa represents the analysis time in minutes.

As shown in FIGS. 1 and 2, lipidic as well as phenolic compounds (semi- to non-volatile compounds) are not detected or are only weakly detected in an essential oil of Santalum album.

### Example 5: Evaluation of the Effect of the Extract of Depleted Sandalwood of Example 1 on the Expression of OR2AT4 Olfactory Receptors in Human Skin Biopsies

The aim of this experiment was to demonstrate an effect of sandalwood extract on the synthesis of the olfactory receptor OR2AT4 in cultured human skin biopsies.

Protocol:

OR2AT4 expression was assessed by indirect immunofluorescence on skin biopsies, pre-treated by topical application of the solubilised extract of depleted sandalwood of Example 1 at 0.1%, 0.5% and 1% (volume/volume percentages), for 48 hours (twice daily). For this purpose, the solubilised extract of depleted sandalwood from Example 1 was diluted in 0.1%, 0.5% and 1% (volume/volume percentages) octyldodecanol. Other biopsies were given similar dilutions of sandalwood essential oil from Example 3 or a synthetic analogue Sandalore. Control biopsies incubated in parallel under the same conditions received placebo (octyldodecanol). After incubation, the biopsies were fixed and embedded in paraffin for histological sections. Detection of the OR2AT4 receptor was performed by incubation with anti-OR2AT4 antibody (Novus). After one and a half hours of incubation followed by rinses, the sections are incubated in the presence of the anti-rabbit secondary antibody coupled to a fluorophore (Alexa Fluor® 488, Invitrogen). The sections were then examined under an Epi-fluorescence microscope (Zeiss Axiovert 200M microscope). OR2AT4 expression was then observed and quantified by image analysis (Volocity® image analysis software, Improvision).

Results:

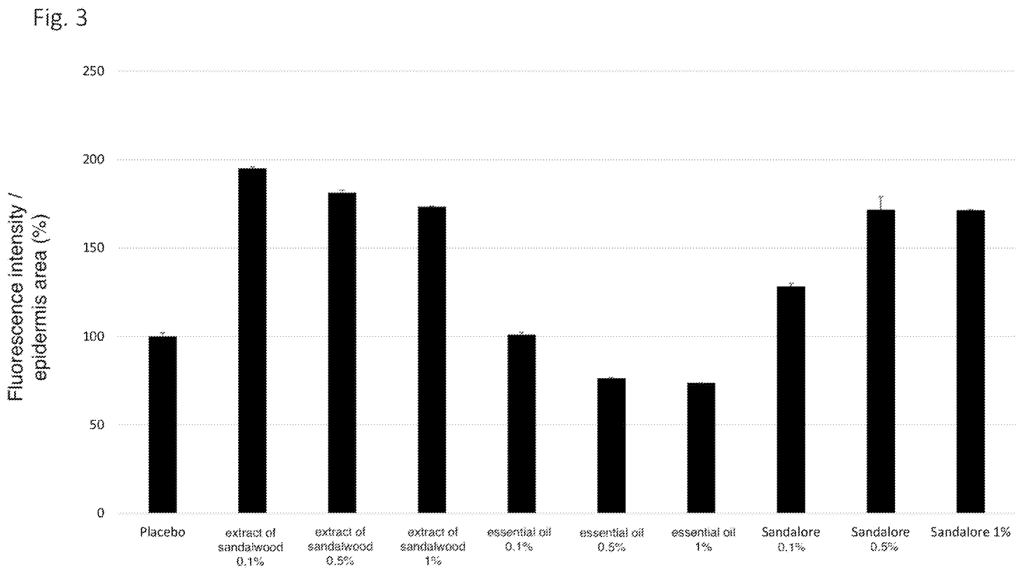

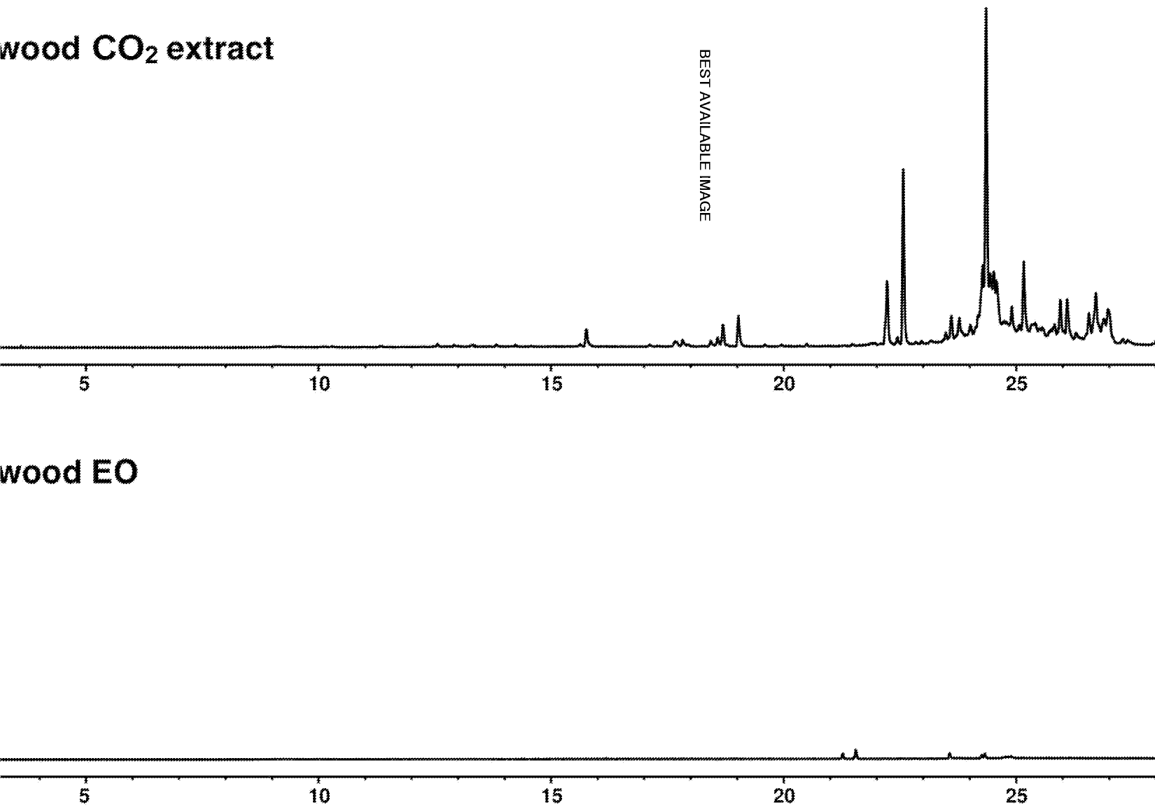

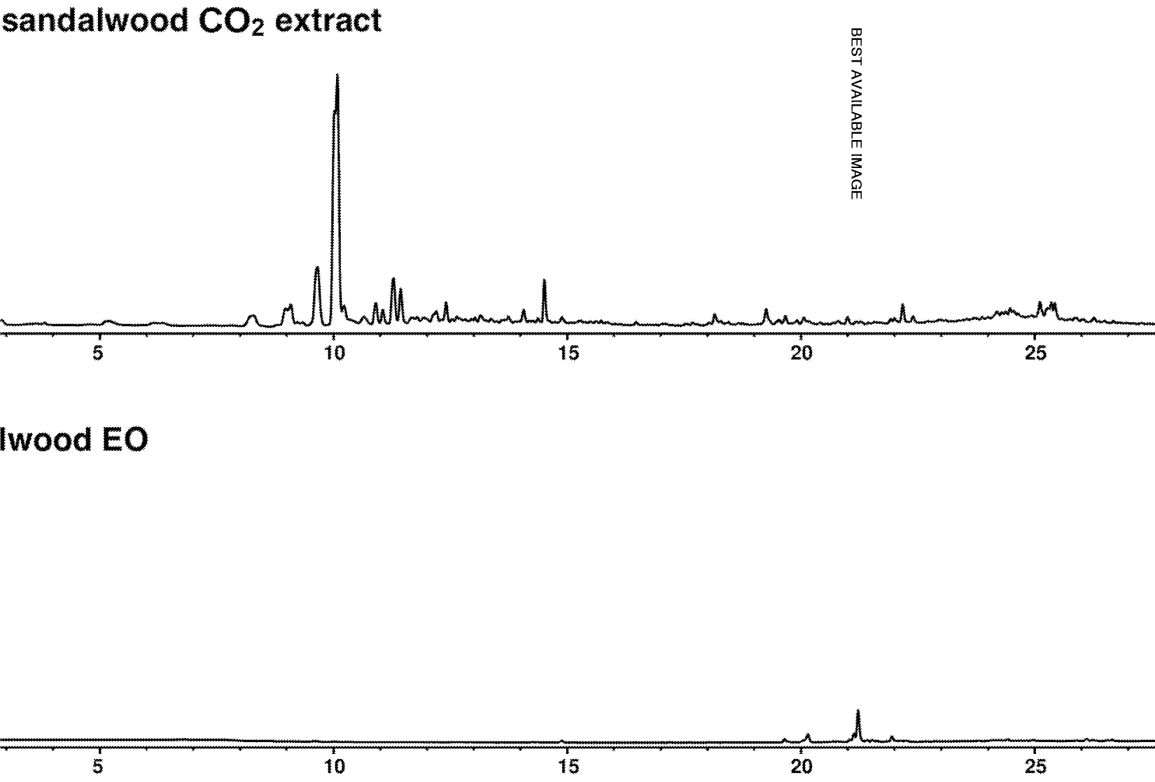

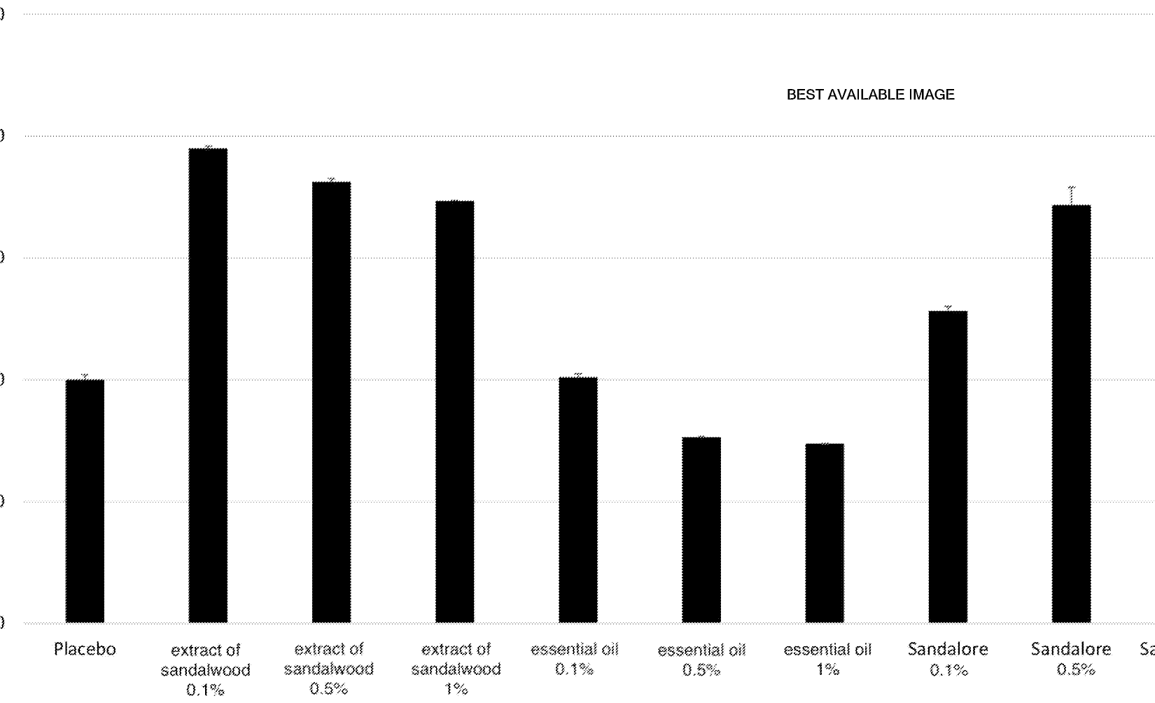

As shown in FIG. 3, when biopsies were treated with sandalwood extract at 0.1%, 0.5% and 1%, OR2AT4 expression increased by 95%, 81% and 73%, respectively. Sandalwood essential oil tested under the same conditions did not induce any increase in OR2AT4 expression and even induced a decrease at 0.5% and 1%. For Sandalore, a known ligand of this receptor, an application of less than 1% does not seem to have any effect on the expression of the receptor. However, following application of Sandalore at 1%, 3% and 5%, OR2AT4 expression was increased by 28%, 72% and 71%, respectively.

Conclusion:

The sandalwood extract showed a positive effect on the expression of the olfactory receptor OR2AT4.

### Example 6: Evaluation of the Effect of the Extract of Depleted Sandalwood of Example 1 on the Homeostasis of the Epidermal Barrier Via the Study of Filaggrin

The aim of this experiment was to demonstrate an effect of sandalwood extract on epidermal barrier homeostasis via the study of filaggrin. Filaggrin is a protein involved in the cohesion and maintenance of the hydration of the upper layer of the epidermis, which is essential for maintaining the homeostasis of the skin barrier.

Protocol:

Filaggrin expression was assessed by indirect immunofluorescence on skin biopsies, pre-treated by topical application of the solubilised extract of depleted sandalwood from Example 1, diluted in 0.1% octyldodecanol (volume/volume percentages, twice daily), for 48 hours. In parallel, biopsies were treated with a sandalwood essential oil of Example 3 from the same geographical site and tested under the same conditions. Control biopsies incubated in parallel under the same conditions were given the placebo (octyldodecanol). After incubation, the biopsies were fixed and embedded in paraffin for histological sections. Detection of filaggrin was performed by incubation with anti-filaggrin antibody (Santa Cruz). After one and a half hours of incubation followed by rinses, the sections were incubated in the presence of the anti-mouse secondary antibody coupled to a fluorophore (Alexa Fluor® 488, Invitrogen). The sections were then examined under an Epi-fluorescence microscope (Zeiss Axiovert 200M microscope). Filaggrin expression was then observed and quantified by image analysis (Volocity® image analysis software, Improvision).

Results:

When biopsies were treated with 0.1% sandalwood extract, filaggrin expression was increased by 28%. Sandalwood essential oil did not induce any increase in filaggrin expression, nor a decrease. In parallel, biopsies treated with a positive control for filaggrin, EGCG (epigallocatechin gallate), showed an increase in filaggrin of +27%.

Conclusion:

The sandalwood extract showed a positive effect on filaggrin expression, indicating an effect on the enhancement of the skin barrier function.

### Example 7: Evaluation of the Effect of the Extract of Depleted Sandalwood of Example 1 on the Lipid Barrier Function Disrupted by Air Pollution Stress Via the Study of Ceramide Synthase 3:

The aim of this experiment was to demonstrate an effect of the sandalwood extract on the homeostasis of the epidermal barrier disrupted by pollution stress via the study of ceramide synthase 3 in cultured human skin biopsies. Ceramide synthase 3 allows the formation of ceramides from sphingosines. Ceramides are members of the sphingolipid family that allow the establishment of barrier function but are also bioactive metabolites involved in epidermal renewal. The stress applied in this experiment was realised with ultrafine particles from diesel engines.

Protocol:

Expression of the enzyme ceramide synthase 3 was assessed by indirect immunofluorescence on skin biopsies, stressed by the application of ultrafine particles from diesel emissions at 500 μg/ml for 16 hours and then treated by topical application of the solubilised extract of depleted sandalwood from Example 1 further diluted in octyldodecanol at 0.1% and 0.5%, and 1% (percentages volume/volume) for 48 hours (twice daily). Control biopsies incubated in parallel under the same conditions received placebo (octyldodecanol). At the end of the incubation, the biopsies were fixed and embedded in paraffin for histological sections. Detection of the enzyme ceramide synthase 3 was performed by incubation with an anti-ceramide synthase 3 antibody (Novus). After one and a half hours of incubation followed by rinses, the sections were incubated in the presence of an anti-rabbit secondary antibody coupled to a fluorophore (Alexa Fluor® 488, Invitrogen). The sections were then examined under an Epi-fluorescence microscope (Zeiss Axiovert 200M microscope). The expression of the ceramide synthase 3 enzyme was then observed and quantified by image analysis (Volocity® image analysis software, Improvision).

Results:

After application of ultrafine diesel particles, ceramide synthase 3 expression was decreased by −39%. When the biopsies were stressed with ultrafine diesel particles and additionally treated with 0.1% and 0.5% sandalwood extract, the expression of ceramide synthase 3 decreased by only −29% and −18%, respectively. After a 1% application of sandalwood extract, the expression level of ceramide synthase 3 even returned to the basal state without stress, indicating a positive effect of sandalwood on this lipid component of the skin barrier.

Conclusion:

Atmospheric ultrafine particle pollution has a negative impact on the expression of ceramide synthase 3. After the application of sandalwood extract, the decrease in the expression of this enzyme is less. Sandalwood extract helps to strengthen the lipid barrier function in the presence of this type of pollutant stress.

### Example 8: Evaluation of the Lightening Potential of the Extract of Depleted Sandalwood of Example 1 on Ex Vivo Skin Biopsies

Principle:

The aim of this study was to evaluate the lightening potential of sandalwood extract on ex vivo skin biopsies, using the Fontana-Masson histological staining of melanin, based on the reduction of an ammoniacal silver nitrate solution to metallic silver. The staining obtained reveals the melanin content and is quantified by image analysis.

Protocol:

Ex vivo human skin biopsies were cultured and treated with the solubilised extract of depleted sandalwood of Example 1 or with an essential oil of sandalwood of Example 3 diluted to 0.1% and 1% (volume/volume percentages) in octyldodecanol for 48 hours. After treatment, the biopsies were fixed for histological analysis and embedded in paraffin. After deparaffinisation, the sections were incubated with ammoniacal silver nitrate solution at 60° C. for 10 minutes. After rinsing, they were treated with 5% Sodium Thiosulphate for 2 minutes, rinsed again, and mounted for examination under the Eclipse E600 microscope (Nikon). The pictures were taken with the QImaging Retiga 2000R Fast1394 camera and analysed with Q-Capture Pro 7 software (QImaging).

Results:

On ex vivo skin biopsies, a decrease in melanin content of −50% and −46% was observed after application of sandalwood extract at 0.1% and 1%, respectively (highly significant by Student's t-test compared to placebo biopsies), while sandalwood essential oil showed smaller decreases of −25% at 0.1% and −21% at 1%.

Conclusion:

This test therefore concluded a potential brightening effect of sandalwood extract on ex vivo skin biopsies.

### Example 9: Evaluation of the Effect of the Extract of Depleted Sandalwood of Example 1 on the Activity of the Dermal Papilla Cells of the Hair Follicle

Principle:

Stimulation of the OR2AT4 olfactory receptors in the hair follicle has been associated with the hair growth phase, called the anagen phase. A marker of this phase is the expression of IGF-1 (insulin-like growth factor 1) in dermal papilla cells. In this example, human dermal papilla cells were treated with the extract of depleted sandalwood of Example 1 and then the expression of IGF-1 was analysed by immunostaining. An increase in this marker is an indicator that the hair follicle remains in the anagen phase.

Protocol:

Human Dermal Papilla Cells (HDPC) were cultured in the presence of the solubilised extract of depleted sandalwood from Example 1, at 0.001 and 0.005% diluted in DMSO. Control cells received an equivalent dilution of DMSO. The treatment was carried out once a day for 48 hours. The cells were then fixed on their support for immunolabelling. They were incubated in the presence of anti-IGF-1 antibody (mouse monoclonal, SantaCruz) overnight. The cells were then rinsed and incubated with the second anti-mouse antibody coupled to the fluorescent marker Alexa Fluor 488 (Invitrogen) for 1 hour. After another washing step, the cells were observed under an Epi-fluorescence microscope (Zeiss Axiovert 200M microscope) and the fluorescence intensity was quantified by image analysis (Volocity® image analysis software, Improvision).

Results and conclusion:

Human dermal papilla cells showed an increase in IGF-1 expression of +27% and +10%, at sandalwood extract concentrations of 0.001% and 0.005%, respectively. This result indicates an activity of sandalwood extract in maintaining the hair growth phase.

### Example 10: Formulation of a Cream for Topical Application

TABLE 4

| | | |
|---|---|---|
| List of ingredients | % | |
| (commercial name/INCI) | (weight/weight) | Provider |

Phase A

| | | | |
|---|---|---|---|
| Purified Water | Water/Aqua | Qs. 100 | Local |
| EDTA tetrasodium Salt | Tetrasodium EDTA | 0.05 | Fisher |
| Lubrajel* MS Free | Glycerin (and) | 3.00 | Ashland |
| hydrogel | Glyceryl | | |
| | Acrylate/Acrylic Acid | | |
| | Copolymer (and) | | |
| | Phenoxyethanol | | |
| LiquaPar ™/Rokonsal ™ | Phenoxyethanol (and) | 1.00 | Ashland |
| MEP preservative | Methylparaben (and) | | |
| | Ethylparaben (and) | | |
| | Propylparaben | | |

Phase B

| | | | |
|---|---|---|---|
| UltraThix ™ P-100 | Acrylic Acid/VP | 0.60 | Ashland |
| polymer | Crosspolymer | | |

Phase C

| | | | |
|---|---|---|---|
| Sodium Hydroxide | Sodium Hydroxide | 0.02 | Fisher |
| Purified Water | Water/Aqua | 0.50 | Local |

Phase D

| | | | |
|---|---|---|---|
| Belsil* W3230 | Bis- | 2.00 | Wacker |
| | Stearoxydimethylsilane | | |
| | (and) Stearyl Alcohol | | |
| | (and) Dimethicone | | |
| Simulsol* 165 | PEG-100 Stearate | 2.00 | Seppic |
| | (and) Glyceryl Stearate | | |
| Refined Shea Butter | Butyrospermum Parkii | 2.00 | Ashland |
| | (Shea) Butter | | |
| Ceraphyl ™ 28 ester | Cetyl Lactate | 1.50 | Ashland |
| Ceraphyl 791 ester | Isocetyl Stearoyl | 2.00 | Ashland |
| | Stearate | | |

What is claimed is:

1. A method for obtaining an extract of sandalwood (Santaluin album) comprising the following steps:
a) adding between 30 and 50% water to the depleted sandalwood;
b) optionally mixing the moistened depleted sandalwood with 1 to 20%, and preferably 5 to 15%, of inert compound;
c) performing an extraction using a fluid in the supercritical state chosen from carbon dioxide (CO₂) and xenon, in the presence of a polar co-solvent chosen from primary or secondary alcohols, or any mixture thereof;
d) evaporating the extract obtained in step c) to remove the co-solvent and thus to recover a crude extract in paste form.

2. The method according to claim 1, wherein in step c) the supercritical fluid is carbon dioxide (CO₂).

3. The method according to claim 1, wherein in step c) the co-solvent is ethanol at a concentration advantageously between 80 and 100% (volume/volume percentage of water), preferably between 90 and 100%, and more preferably ethanol is used at a concentration of 96% (volume/volume percentage of water).

4. The method according to claim 1, wherein in step c) the weight ratio of supercritical fluid (carbon dioxide) to co-solvent is between 0.050 and 0.080, advantageously between 0.055 and 0.075, and preferably between 0.060 and 0.070.

5. The method according to claim 1, wherein the ratio by weight, of the supercritical fluid to the amount of depleted sandalwood used is between 10 and 50, advantageously between 20 and 40, and preferably between 25 and 35.

6. The method according to claim 1, wherein in step c) the extraction temperature is between 35 and 85° C., advantageously between 45 and 75° C., and preferably between 55 and 65° C., and the pressure within the extractor is between 90 and 1000 bar, preferably between 150 and 700 bar, and even more preferably between 250 and 400 bar.

7. The method according to claim 1, wherein the crude extract obtained in step d) is solubilised in a saturated or unsaturated, linear or branched, fatty alcohol solvent comprising 8 to 30 carbons or a glyceride solvent, or any mixture thereof, the solvent preferably being selected from octyldodecanol, 2-hexyl decanol, oleic alcohol, and a mixture of triglycerides, to obtain a concentration of crude extract in the final extract, of between 0.5 and 1.5% by weight in relation to the total weight of the solubilised extract.

8. A crude sandalwood extract obtainable by the method according to claim 1, characterised in that it comprises from 10 to 70%, preferably from 12 to 23%, more preferably from 14 to 21%, and even more preferably from 16 to 19% of volatile compounds, and from 30 to 90%, preferably from 77 to 88%, more preferably from 79 to 86%, and even more preferably from 81 to 84% of semi- to non-volatile compounds.

9. The crude sandalwood extract according to claim 8, characterised in that it comprises, among the semi- to non-volatile compounds, between 0.35 and 3.5% syringaldehyde, advantageously between 0.4 and 3%, and preferably between 0.45 and 2.5%.

10. A solubilised sandalwood extract obtainable by the method according to claim 7, characterised in that it comprises, among the semi- to non-volatile compounds, between 0.0035% and 0.035% syringaldehyde, and in that the solubilising solvent is octyldodecanol.

11. A composition comprising as active agent a solubilised extract of depleted sandalwood according to claim 10, at a concentration of between 0.001 and 1%, preferably between 0.1 and 1%, and a physiologically acceptable medium.

12. A method of cosmetic treatment comprising applying to the skin a composition according to claim 11 for care of the skin, scalp and appendages.

13. The method of cosmetic treatment according to claim 12 to improve the appearance of the skin, fight the signs of skin ageing, improve the skin's ability to detect external pollutants, and restore the skin's lipid barrier function.

14. The method of cosmetic treatment 12 to increase the expression of olfactory receptors (OR2AT4) in the skin.

15. The method of cosmetic treatment according to claim 12 to lighten the skin.

16. The method of cosmetic treatment according to claim 12 to promote hair growth.
